(19) United States
(12) Patent Application Publication
(10) Pub. No.: US 2003/0134825 A1
(43) Pub. Date: Jul. 17, 2003

(54) Food product supplemented with proteoglycan precursors

(76) Inventor: Robert Bahoshy, Long Beach, CA (US)

Correspondence Address: Peng Chen, Morrison & Foerster LLP, Suite 500, 3811 Valley Centre Drive, San Diego, CA, 92130-2332, US

(21) Appl. No.: 10/336,652

(22) Filed: Jan. 3, 2003

Related U.S. Application Data
(60) Provisional application No. 60/344,053, filed on Jan. 3, 2002.
(60) Provisional application No. 60/353,051, filed on Jan. 29, 2002.

(51) Int. Cl. A61K 31/7008; A61K 47/00

(52) U.S. Cl. 514/62; 424/439

(57) ABSTRACT

A food product for supplementing the proteoglycan precursor intake of humans suffering from joint related ailments and a method of making and administering such a food product is disclosed. More specifically, a settable food product, supplemented with glucosamine and chondroitin, which comes in a suitable form, e.g., a pudding or pie filling, is disclosed, along with a method of making and administering the product to persons in need thereof.

This application claims the benefit of Provisional Patent Application No. 60/344,053 filed on Jan. 3, 2002, and Provisional Patent Application No. 60/353,051 filed on Jan. 29, 2002 under 35 U.S.C. §119(e). The contents of the above-referenced applications are incorporated herein in their entireties.

Throughout this application, various references are referred to. Disclosures of these publications in their entireties are hereby incorporated by reference into this application to more fully describe the state of the art to which this invention pertains.

### BACKGROUND INVENTION

1. Field of the Invention

The present invention generally relates to food compositions for the treatment of joint related ailments, and methods for making and administering these compositions. In particular, the present invention relates to the preparation of compositions including proteoglycan precursors and method for administering these precursors in a beneficial and appetizing manner to persons in need thereof.

2. Description of the Related Art

Millions of people suffer from the debilitating effects of joint related ailments. Of particular interest in this area are the ailments related to arthritis. Among the many types of arthritis, osteoarthritis is the most prevalent, especially among the elderly. Osteoarthritis is associated with a breakdown of cartilage that commonly occurs in the joints such as, hips, knees, fingers, feet and spine. Over time, the cartilage may wear away in some areas greatly decreasing its effectiveness, even to the point where bones may rub directly against each other. Conventional treatments for osteoarthritis include medication, exercise, diet and applying heat and cold to the pain afflicted areas. None of these common treatments alter the progression of osteoarthritis. Among medications prescribed to address this illness, non-steroidal anti-inflammatories (NSAIDs) are the most common. Unfortunately, these medications have a number of side effects and may even increase the progression of osteoarthritis. Other forms of joint related ailments exist due to the everyday stress placed on these connective tissues.

Over the past two decades, an alternative treatment for joint related ailments has emerged. The alternative treatment involves administering glucosamine and chondroitin supplements to patients suffering from joint related ailments. These two proteoglycan precursors represent a proactive treatment for treating and maintaining joint health. Recently they have demonstrated pain relief effects in arthritic patients and may even reverse the effects of arthritis and assist the body to repair and rehabilitate damaged cartilage. Unlike other medications, they have no known side effects.

Glucosamine and chondroitin are components of normal cartilage. Both act as precursors in the formation of proteoglycans which in turn become the building blocks of connective tissue. While glucosamine is a multifunctional precursor of proteoglycan synthesis in general and glycosaminoglycans in particular, chondroitin is a glycosaminoglycan that is preferentially incorporated into cartilaginous tissue. Because of its tropism for cartilage, chondroitin is the most abundant glycosaminoglycan in cartilage and is responsible for the resiliency of joint tissue.

While the body normally generates enough proteoglycan precursors to maintain levels of cartilage throughout, many people suffering from arthritis require supplements of these very important compounds. However, it is difficult to supplement their intake merely by a change in diet because the sources of glucosamine and chondroitin are not commonly found in foods. In particular, glucosamine is derived and isolated from chitin. Chitin is a major component of the shells of sea animals such as crab and sea shrimp. Edible chondroitin on the other hand is derived from animal connective tissue such as tendons, cartilage and trachea. Because of the difficulty of including these items in a normal diet, glucosamine and chondroitin commonly require administration through oral supplements. Common oral supplements take the form of capsules, tablets or pills. Similar supplements are disclosed in U.S. Pat. No. 6,255,295, U.S. Pat. No. 6,162,787, and U.S. Pat. No. 5,840,715, among others. These types of delivery methods often fail because many people have difficulty taking pills, dislike taking them or forget to take enough to meet the effective dosage. Of particular interest is the elderly community, which commonly suffers from difficulties ingesting foods and nutrients. Many of their medications must be administered via liquid diets and/or intravenously. The following invention seeks to solve these problems by incorporating proteoglycan precursor supplements into a desirable food product that may be easily ingested by both young and old, as well as those incapable of adhering to a solid diet.

Information relevant to attempts to address these problems can be found in U.S. Pat. No. 5,922,692. This reference generally discloses methods of manufacturing glucosamine and chondroitin to be added to foodstuffs. However, this reference suffers from the disadvantage of a final product that simply adds the chondroitin and glucosamine to foodstuffs without consideration of the taste characteristics encountered by the consumer, or whether these ingredients may affect the final products' physical attributes. In particular, the formulations that include glucosamine and chondroitin do not take into account the effect these supplements have on the taste of a food product and fail to address the need to make the product more appealing to human consumers.

For the foregoing reasons, there is a need for a simple, inexpensive, lightweight and easily ingestible food product which consumers will enjoy eating. In addition, that food product must take into account the special problems created by the addition of proteoglycan precursors to food in order for the product to appeal to consumers while maintaining its physical attributes.

### SUMMARY OF THE INVENTION

Due to the existing need for a product that supplements chondroitin and/or glucosamine intake by persons in need of such supplementation, a brief summary of the present invention is presented. Some simplifications and omissions may be made in the following summary, which is intended to highlight and introduce some aspects of the present invention, but not limit its scope. Detailed descriptions of a preferred exemplary embodiment adequate to allow those of ordinary skill in the art to make and use the invention concepts will follow in later sections.

A settable food product is disclosed which generally comprises a natural or synthetic non-gelatin gelling agent with the addition of a proteoglycan precursor and a liquid, which is characterized by its ability to set the combined food product.

Additionally, a method is disclosed for making a settable food product by providing a non-gelatin gelling agent along with a proteoglycan precursor and a liquid having an ability to set the combined food product. The composition is then combined to produce an edible food product.

Further, a method of administering a food product containing a proteoglycan precursor is disclosed. An easily ingested food product containing proteoglycan precursors, a non-gelatin gelling agent and a liquid capable of setting these ingredients is prepared with sufficient proteoglycan precursors to supplement the diet of a person in need thereof. The food product is then administered either in one dosage, or in multiple dosages.

### DESCRIPTION OF THE INVENTION

One embodiment of the present invention comprises a product designed to help consumers supplement their diet with proteoglycan precursors. In particular, the invention is a settable food product fortified with proteoglycan precursors. Two proteoglycan precursors, glucosamine and chondroitin may be major components of the composition. As described above, glucosamine and chondroitin have shown pain relieving and other beneficial qualities, especially in the treatment of joint related ailments.

The settable food product composition will generally include a natural or synthetic non-gelatin gelling agent, one or more proteoglycan precursors, a liquid, and additives affecting the taste and appearance of the product. These additives may include, but are not limited to, edible acids, buffers, sweeteners, natural and artificial flavors and coloring agents.

The settable food product may take a variety of forms. The food product may be sold in ready to eat forms or comprise a dry mix that requires preparation by the consumer. The settable food product may include but is not limited to any one of the following: a ready to eat or pudding dry mix, pudding, instant pudding, pie filling, or pie filling mix.

Gelling agents are used to help set the food product after it has been dissolved in a liquid. Natural or synthetic gelling agents may include but are not limited to starch, and pregelatinized modified starch.

There are a number of proteoglycan precursors that may be used in the composition, either alone or in combination. One such precursor is glucosamine and effective salts thereof. This may include, but is not limited to, chitosamine, glucosamine sulfate, glucosamine hydrochloride, glucosamine iodide, and N-Acetylglucosamine, and mixtures thereof. Another such precursor is chondroitin 4-sulfate, chondroitin 6-sulfate and chondrosine, and mixtures thereof. The amount of proteoglycan precursors must be carefully measured in order to achieve the desired flavor and settable characteristics of the food product.

In addition, this invention also includes the above composition which further includes an appropriate amount of haluronic acid. Haluronic acid is well-known in the art.

A number of edible acids may be used in the composition. As shown in U.S. Pat. No. 2,519,961, edible acids control the proper pH of the product and add a desired tart taste. These edible acids may include, but are not limited to citric acid, adipic acid, tartaric acid, ascorbic acid, isoascorbic acid, malic acid, and erythorbic acid, and mixtures thereof.

A buffer salt may also be included in order to modify the pH, the setting and the melting characteristics of the food product. Such buffer salts include but are not limited to citrates, tartrates, phosphates and pyrophosphates.

Both natural and synthetic sweeteners may be used in the food product. Sweeteners add taste to the product and allow it be eaten as a dessert. Also, sweeteners may be required to modify the flavor effects of the proteoglycan precursors in the food product. Natural sweeteners may include, but are not limited to sucrose, glucose, fructose, mannitol, dextrose, and mixtures thereof. Artificial sweeteners may include, but are not limited to, saccharin, aspartame, and acesulfame, and mixtures thereof.

A number of other additives may be added to modify the taste, color, texture, or other factors that affect consumer appeal of the food product.

An exemplary cold to ambient environment has a temperature ranging from about 4° C. to about 30° C.

For purposes of describing embodiments of the present invention, examples are provided to further illustrate the invention.

### EXAMPLE 1

A naturally or artificially flavored dry powder pudding or pie filling mix is prepared with the following ingredients:

TABLE 1

Dry Mix
Grams Per Serving

| | Range | Preferred |
|---|---|---|
| Sugar | 15-25 | 20 |
| Modified Starch | 20-30 | 25 |
| Natural & Artificial Flavors | 1.5-2.5 | 2 |
| Salt | 0.05-0.15 | 0.1 |
| Mono Diglycerides | 0.1-0.2 | 0.15 |
| Color Yellow #5 | 0.001 | 0.001 |
| Color Yellow #6 | 0.001 | 0.001 |
| Carrageenan Gum | 0.5-1.0 | 0.5 |
| Glucosamine | 0.5-3.0 | 0.75 |
| Chondroitin Sulfate | 0.4-2.4 | 0.60 |
| Milk | 0.0 | 0.0 |
| | Total | 49.102 |

The above ingredients are prepared in the following manner. Sugar and all ingredients except for starch are blended for 5 minutes, starch is added and blended for 15 minutes. Sugar can be replaced by artificial sweeteners such as aspartame and acesufame-K for the purpose of making a sugarless food product. Natural and artificial flavors can include vanilla, chocolate, coconut or various fruit flavors. The milk can be non-fat, 1%, 1½%, 2%, whole milk or a non-dairy milk equivalent. The color can also be varied as desired. Once the dry mix is prepared, it is packaged for consumer use. For multiple servings, multiply the single serving amount by the desired servings. The recipe for consumer use further states: add 120 grams of milk to the powder mix and heat to a boil, and then refrigerate the product until cool.

### EXAMPLE 2

A naturally or artificially flavored pudding or pie filling mix in ready to eat form is prepared with the following ingredients:

TABLE 2

Ready to Eat
Grams Per Serving

| | Range | Preferred |
|---|---|---|
| Sugar | 15-25 | 20 |
| Modified Starch | 20-30 | 25 |
| Natural & Artificial Flavors | 1.5-2.5 | 2 |
| Salt | 0.05-0.15 | 0.1 |
| Mono Diglycerides | 0.1-0.2 | 0.15 |
| Color Yellow #5 | 0.001 | 0.001 |
| Color Yellow #6 | 0.001 | 0.001 |
| Carrageenan Gum | 0.5-1 | 0.5 |
| Glucosamine | 0.5-3.0 | 0.75 |
| Chondroitin Sulfate | 0.4-2.4 | 0.60 |
| Milk | 100-140 | 120 |
| | Total | 169.102 |

The above ingredients are prepared in the following manner. All ingredients are added to cold milk and agitated at a high level for 5 minutes. Heat mixture to 280 degrees Fahrenheit, then cool to 900-100 degrees Fahrenheit and pack for consumer use. Sugar can be replaced by artificial sweeteners such as aspartame and acesufame-K for the purpose of making a sugarless food product. Natural and artificial flavors can include vanilla, chocolate, coconut or various fruit flavors. The milk can be non-fat, 1%, 1½%, 2%, whole milk or a non-dairy milk equivalent. The color can also be varied as desired. For multiple servings, multiple the single serving amount by the desired servings.

### EXAMPLE 3

A naturally or artificial flavored pudding or pie filling mix with a very short preparation time similar to “instant pudding” is prepared with the following ingredients:

TABLE 3

Instant Pudding
Grams Per Serving

| | Range | Preferred |
|---|---|---|
| Sugar | 15-25 | 20 |
| Pregelatinized Modified Starch | 3.0-4.0 | 3.5 |
| Natural and Artificial Flavor* | 1.5-2.5 | 2 |
| Salt | 0.05-0.15 | 0.1 |
| Disodium Phosphate | 0.3-0.5 | 0.4 |
| Tetrasodium Pyrophosphate | 0.3-0.5 | 0.4 |
| Mono Diglycerides | 0.1-0.2 | 0.1 |
| Color Yellow #5 | 0.001 | 0.001 |
| Color Yellow #6 | 0.001 | 0.001 |
| Glucosamine | 0.5-3.0 | 0.75 |
| Chondroitin Sulfate | 0.4-2.4 | 0.60 |
| | Total | 27.852 |

The above ingredients are prepared in the following manner. All ingredients except starch are blended for 5 minutes. Starch is then added and the mixture is blended for 15 minutes. Glucosamine and Chondroitin levels must be limited below 1.5 and 1.2 grams respectively due to the salty taste that occurs above those levels. Sugar can be replaced by artificial sweeteners such as aspartame and acesufame-K for the purpose of making a sugarless food product. Natural and artificial flavors can include vanilla, chocolate, coconut or various fruit flavors. The color can also be varied as desired. Once the dry mix is prepared it is packaged for consumer use. For multiple servings, multiply the single serving amount by the desired servings. The recipe for consumer use further states: blend powder mix with ½ cup cold milk for 2 minutes, then refrigerate for 5-10 minutes. The milk can be non-fat, 1%, 1½%, 2%, whole milk or a non-dairy milk equivalent.

What is claimed is:

1. A settable food product, comprising:
a natural or synthetic gelling agent, wherein said gelling agent is not gelatin;
one or more proteoglycan precursors; and
a liquid characterized by its ability to set the combined food product.

2. A food product according to claim 1, wherein the proteoglycan precursor is glucosamine and effective salts thereof.

3. The food product of claim 2, wherein each serving comprises 1-3,000 mg of glucosamine and effective salts thereof.

4. The food product of claim 1, wherein the proteoglycan precursor is chondroitin and effective salts thereof.

5. The food product of claim 4, wherein each serving comprises 1-2,400 mg of chondroitin and effective salts thereof.

6. The settable food product of claim 1, wherein the proteoglycan precursors are glucosamine and effective salts thereof and chondroitin and effective salts thereof.

7. The settable food product of claim 6, wherein each serving comprises 1-3,000 mg of glucosamine and effective salts thereof and 1-2,400 mg of chondroitin and effective salts thereof in each serving.

8. The settable food product of claim 1, further comprising a sweetening agent.

9. The food product of claim 8, wherein the sweetening agent is natural, synthetic or combinations thereof.

10. The food product of claim 9, wherein the sweetening agent comprises sucrose, fructose, mannitol, glucose, dextrose, saccharin, aspartame, or acesulfame-K, and mixtures or combinations thereof.

11. The food product of claim 1, wherein at least one or more of the gelling agents, and the proteoglycan precursor, are in powder form prior to combining with the liquid.

12. The food product of claim 1, further comprising an acid.

13. The food product of claim 12, wherein the acid comprises citric acid, adipic acid, fumaric acid, malic acid, or erythorbic acid or mixtures thereof.

14. The food product of claim 1, wherein the food product is pudding.

15. The food product of claim 1, wherein the food product is pie filling.

16. A method for making a settable food product comprising:
combining a mixture of a natural or synthetic gelling agent, wherein said gelling agent is not gelatin;
one or more proteoglycan precursors;
a sweetener; and
an edible acid.

17. The method of claim 16, wherein the proteoglycan precursor is glucosamine and effective salts thereof.

18. The method of claim 16, wherein each serving of the settable food product comprises 1-3,000 mg of glucosamine and effective salts thereof.

19. The method of claim 16, wherein the proteoglycan precursor is chondroitin and effective salts thereof.

20. The method of claim 19, wherein each serving of the settable food comprises 1-2,400 mg chondroitin and effective salts thereof.

21. The method of claim 20, wherein the mixture dissolves in water.

22. The method of claim 21, wherein the dissolved mixture sets when placed in a cold to ambient environment.

23. The method of claim 20, wherein the mixture dissolves in milk.

24. The method of claim 23, wherein the dissolved mixture sets in a cold to ambient environment.

25. A method for making a settable food product, comprising the steps of:
providing a gelling agent, wherein said gelling agent is not gelatin;
providing a proteoglycan precursor;
providing a liquid characterized by its ability to set the combined food product; and combining to produce a food product.

26. The method of claim 25, wherein the proteoglycan precursor is glucosamine and effective salts thereof.

27. The method of claim 26, wherein each serving of the settable food product comprises 1-3,000 mg of glucosamine and effective salts thereof.

28. The method of claim 25, wherein the proteoglycan precursor is chondroitin and effective salts thereof.

29. The method of claim 28, wherein each serving of the settable food product comprises 1-2,400 mg of chondroitin and effective salts thereof.

30. The method of claim 25, wherein the liquid is water.

31. The method of claim 30, wherein the mixture dissolves in water.

32. The method of claim 31, wherein the dissolved mixture sets when placed in a cold to ambient environment.

33. The method of claim 25, wherein the liquid is milk.

34. The method of claim 33, wherein the mixture dissolves in milk.

35. The method of claim 34, wherein the dissolved mixture sets when placed in a cold to ambient environment.

36. The food product of claim 1 further comprising a haluronic acid.

37. The method of claim 25, further comprising the step of providing a haluronic acid.
